(19) United States
(12) Patent Application Publication
(10) Pub. No.: US 2017/0259256 A1
(43) Pub. Date: Sep. 14, 2017

(54) REGENERATION OF WEAK BASE ANION EXCHANGE RESINS

(71) Applicant: Purolite Corporation, Bala Cynwyd, PA (US)

(72) Inventors: William Fries, Southhampton, PA (US); Carmen Mihaela Iesan, Fagaras (RO); Robert Moore, Belvidere, IL (US)

(21) Appl. No.: 15/504,822

(22) PCT Filed: Aug. 14, 2015
(86) PCT No.: PCT/US2015/045356

§ 371 Date: Feb. 17, 2017

Related U.S. Application Data
(60) Provisional application No. 62/039,266, filed on Aug. 19, 2014.

(51) Int. Cl. B01J 49/57; B01D 15/36; A23L 5/20; A23L 27/30; C07C 51/47; C13B 20/14

(52) U.S. Cl. CPC B01J 49/57; C07C 51/47; C13B 20/146; A23L 5/273; A23L 27/33; A23L 27/35; A23L 27/34; B01D 15/363; A23V 2002/00

(57) ABSTRACT

The present invention relates generally to regeneration of weak base anion exchange resins and more particularly to regeneration using low concentrations of sodium hydroxide and/or sodium carbonate to remove ionic contaminants from the resins.

### FIELD OF THE INVENTION

The present invention relates generally to regeneration of ion exchange resins and more particularly to regeneration using low concentrations of a fluid regenerant solution having a basic pH to remove ionic contaminants from the resins.

### BACKGROUND OF THE INVENTION

Weak base anion exchange resins (“WBA” resins) typically include primary (R—NH₂), secondary (R—NHR′), or tertiary (R—NR′₂) amine group functionality. WBA resins readily remove a broad array of ionic impurities (including sulfuric, nitric, phosphoric, hydrochloric and amino acid contaminants) from a variety of organic feedstocks (including acetic acid, formic acid, citric acid, succinic acid, lactic acid and glycolic acid) and saccharides (such as glucose syrup, dextrose, 42% high fructose corn syrup (HFCS)), polyols (such as hydrogenated sweeteners) and gelatin. WBA resins are also used to remove acidic impurity components from beverages including water, fruit juices and dairy products. WBA resins are generally provided as spherical beads, having an average diameter of less than 1200 microns. As used herein, reference to “resins” generally means resin provided in bead form, although other physical forms of WBA resin may be employed, such as granular resins.

WBA resins are initially hydrophobic (in the free base (FB) form) but become progressively more hydrophilic in use, as anion exchange takes place and they become exhausted, such as when loaded with sulfuric acid, nitric acid, phosphoric acid and other ionic contaminants. The latter form is ionized; the former is not. As a result, the amount of water of hydration increases markedly from the former to the latter. This also means that the resin must swell markedly to accommodate the water.

Uneven swelling of the resin can place excessive stress upon the resin structure. WBA resins are typically provided in generally spherical bead form. During use, the shell, or outer portion of the bead, becomes ionized and therefore more hydrophilic and hydrated (swollen). At the same time the core, or inner portion of the bead has not yet become ionized and remains hydrophobic and does not swell. The transition zone interface between the core and the swollen shell is subject to shear forces. This effect is sometimes called “osmotic shock”. During use to remove contaminants from the feedstock, the ion exchange occurs at a relatively low rate, such that the disequilibrium between the swelling of the shell and the core is minimized However, when the resin is exhausted (when substantially all exchange sites within the bead have been exchanged with ionic contaminants), the resin must be regenerated. Typically, regeneration of a WBA resin takes place by subjecting the resin to treatment with a strongly basic liquid solution such as sodium hydroxide. Regeneration of the resin to remove the ionic contaminants from the exhausted beads requires exposure to at least a stoichiometric amount of base. To maintain a high rate of regeneration, it is conventional to apply a high concentration of sodium hydroxide (e.g., a 4-5% (weight per volume, w/v) NaOH solution) at a flow rate, for example, of 2 bed volumes per hour for a period of 45-75 minutes. While such treatment can regenerate the resin quickly, it also results in uneven expansion forces applied to different parts of the bead. The regeneration proceeds heterogeneously, as the outer shell is converted to the hydrophobic form and the core remains hydrophilic. The newly formed hydrophobic shell shrinks in size and becomes more dense, inhibiting the migration of the sodium hydroxide regenerant solution into the hydrophilic core. The resulting forces can be strong enough to cause cleavage or fracturing of the bead, resulting in the generation of undesirable fines. Fines reduce capacity, can cause clogging and increased hydrostatic pressure on the resin bed, reducing throughput. At the same time, the inability to penetrate to the core with regenerant also results in less complete regeneration, and therefore, lower operating capacity of the regenerated resin beads.

Although regeneration of WBA resins with a solution of sodium hydroxide at a concentration of 4% (w/v) or more at high flow rates can result in rapid regeneration times, at the same time the process results in deterioration of the resin beds due to fracture and generation of fines. Fractured beads can cause clogging and increased hydrostatic pressure on the resin bed. A process that can regenerate WBA resins without significant bead fracture and fines generation, and restore a high proportion of the operating capacity of the resin, would be highly desirable.

### SUMMARY OF THE INVENTION

According to an embodiment of the present invention, a method of regenerating a weak base anion exchange resin is provided. The method includes providing a weak base anion exchange resin, at least partially bound to ionic contaminants. The resin is contacted with a regenerant solution including a base selected from the group consisting of sodium hydroxide, sodium carbonate and mixtures thereof, whereby at least a portion of said ionic contaminants are unbound from said resin. The resin is then rinsed to remove said ionic contaminants. In some embodiments, the base is sodium hydroxide and is provided at a concentration of 3% or less, 2% or less, 1% or less, 0.5% or less or 0.25% or less. In some embodiments, the base is sodium carbonate and is provided at a concentration of 3% or less, 2% or less, 1% or less, 0.5% or less or 0.25% or less.

### BRIEF DESCRIPTION OF THE DRAWINGS

FIG. 1 is a graph depicting the percentage of intact resin beads remaining after repeated treatment cycles of an acrylic resin with sodium hydroxide regenerant solutions of different concentrations.

FIG. 2 is a graph depicting the percentage of intact resin beads remaining after repeated treatment cycles of a styrenic resin with sodium hydroxide regenerant solutions of different concentrations.

FIG. 3 is a graph depicting the percentage of intact resin beads remaining after repeated treatment cycles of a styrenic resin with sodium carbonate regenerant solutions of different concentrations.

### DETAILED DESCRIPTION

The present invention is based on the determination that regeneration of an exhausted WBA resin using much lower concentrations of regenerant than conventional methods, and optionally conducting the regeneration at a lower rate than conventional methods, results in regeneration of the resin with less breakage of resin beads and lower fine generation. In addition, it has been discovered that regeneration under such conditions can also restore a high proportion of the operating capacity of the resin.

The resin used in the process of the invention can include a weak base anion (WBA) exchange resin, resins including a polystyrene acrylic (optionally cross-linked with divinylbenzene), or a phenol formaldehyde matrix structure. Gel-type and macroporous anion exchange resins are included within the scope of the invention. The term “ion exchange resin” is intended to broadly describe polymer resin particles which have been chemically treated to attach or form functional groups which have a capacity for ion exchange and acid adsorption. The term “functionalize” refers to processes (e.g. sulfonation, haloalkylation, amination, etc.) for chemically treating polymer resins to attach ion exchange groups, i.e. “functional groups.” The polymer component serves as the substrate or polymeric backbone whereas the functional group serves as the active site capable of exchanging ions with a surrounding fluid medium. The present invention also includes a class of ion exchange resins comprising cross-linked copolymers including interpenetrating polymer networks (IPN). The term “interpenetrating polymer network” is intended to describe a material containing at least two polymers, each in network form wherein at least one of the polymers is synthesized and/or cross-linked in the presence of the other polymer. The polymer networks are physically entangled with each other and in some embodiments may be also be covalently bonded. Characteristically, IPNs swell but do not dissolve in solvent nor flow when heated. Ion exchange resins including IPNs have been commercially available for many years and may be prepared by known techniques involving the preparation of multiple polymer components.

As used herein, the term “polymer component” refers to the polymeric material resulting from a polymerization reaction. For example, in one embodiment of the present invention, the ion exchange resins are “seeded” resins; that is, the resin is formed via a seeded process wherein a polymer seed is first formed and is subsequently treated with monomer and subsequently polymerized. Additional monomer may be subsequently added during the polymerization process. The monomer mixture used during a polymerization step need not be homogeneous; that is, the ratio and type of monomers may be varied. The term “polymer component” is not intended to mean that the resulting resin have any particular morphology. Examples of suitable crosslinking agents include monomers such as polyvinylidene aromatics such as divinylbenzene, divinyltoluene, divinylxylene, divinylnaphthalene, trivinylbenzene, divinyldiphenyl ether, divinyldiphenylsulfone, as well as diverse alkylene diacrylates and alkylene dimethacrylates. Preferred crosslinking monomers are divinylbenzene, trivinylbenzene, and ethylene glycol dimethacrylate. The terms “crosslinking agent,” “crosslinker” and “crosslinking monomer” are used herein as synonyms and are intended to include both a single species of crosslinking agent and combinations of different types of crosslinking agents.

The polymer particles of the present invention can also be prepared by suspension polymerization of an organic phase comprising, for example, monovinylidene monomers such as styrene, crosslinking monomers such as divinylbenzene, a free-radical initiator and, optionally, a phase-separating diluent. The polymer may be macroporous or gel-type. The terms “gel-type” and “macroporous” are well-known in the art and generally describe the nature of the copolymer particle porosity. The term “macroporous” as commonly used in the art means that the copolymer has both macropores and mesopores. The terms “microporous,” “gellular,” “gel” and “gel-type” are synonyms that describe polymer particles having pore sizes less than about 20 Angstroms while macroporous polymer particles have both mesopores of from about 20 to about 500 Angstroms and macropores of greater than about 500 Angstroms. In some embodiments, the macroporous resin of the invention has a pore diameter range of 500-100,000 Angstroms, and the specific volume of the pores ranges from 0.5-2.1 cc/g.

The term “anion-exchange resin” indicates a resin which is capable of exchanging negatively charged species with the environment. The term “strong base anion exchange resin” refers to an anion exchange resin that comprises positively charged species which are linked to anions such as Cl⁻, Br⁻, F⁻ and OH⁻. The most common positively charged resin functionalization species are quaternary amines and protonated tertiary amines. Suitable anion-exchange resins include resins whose matrix is either hydrophilic or hydrophobic including anion-exchange resins wherein the exchanging groups are strongly or weakly basic in either gel or macroporous forms. Preferably, the matrix is polystyrene or polyacrylic, gel form, particularly based on polystyrene/divinylbenzene copolymer. Anion exchange resins may include strong base anion exchange resins (SBA), weak base anion exchange resins (WBA) and related anionic functional resins, of either the gelular or macroporous type containing quaternary ammonium functionality (chloride, sulfate, hydroxide or carbonate forms), dialkylamino or substituted dialkylamino functionality (free base or acid salt form), and aminoalkylphosphonate or iminodiacetate functionality, respectively.

The present invention is particularly applicable to using weak base anion (WBA) exchange resins. Weak base resin functionality typically includes primary (R—NH₂), secondary (R—NHR′), or tertiary (R—NR′₂) amine groups. WBA resins readily remove acidic impurities including sulfuric, nitric, hydrochloric and phosphoric acids from a variety of feedstocks containing such acids and from which removal of such acids is desired. Such feedstocks include acetic acid, formic acid, citric acid, succinic acid, lactic acid and glycolic acid and starch-based sweeteners such as glucose syrup, dextrose, 42% HFCS, hydrogenated sweeteners (polyols), cellulose hydrolyzate and gelatin. Weak functionality resins generally have a higher regeneration efficiency than their strong functionality counterparts. In some embodiments, the anion exchange resin is a Purofine® PFA847 resin, a weak base gel-type anion exchange resin with an acrylic matrix, available from Purolite Corporation, Bala Cynwyd, Pa.

Examples of other weak base gel-type anion exchange resins that are useful in the invention include Purolite® A845, Purolite® A845DL, Purolite® A847C, Purolite® A847DL, Purolite® A847S, and Puropack® PPA847 resins, also available from Purolite Corporation, Bala Cynwyd, Pa.

In some embodiments, the anion exchange resin is a Purofine® PFA133SPlus, Purofine® PFA103SPlus or Purofine® PPA103SPlus resin, a weak base macroporous anion exchange resin with a polystyrene matrix structure. Another suitable polystyrene gel type resin is Purolite® A172/4635, also available from Purolite Corporation, Bala Cynwyd, Pa.

Other macroporous weak base anion exchange resins include, but are not limited to, Purolite® A100CPlus/4317, Purolite® A100DLPlus, Purolite® A100DRPlus, Purolite® AlOOINDPlus, and Purolite® AlOOSPlus, each available from Purolite Corporation, Bala Cynwyd, Pa.

In some embodiments, the ion exchange resin is a weak base anion exchange resin.

In some embodiments, the weak base anion exchange resin is a gel-type anion exchange resin comprising an acrylic matrix.

In some embodiments, the acrylic matrix structure is cross-linked with divinylbenzene.

In some embodiments, the weak base anion exchange resin is a macroporous resin with a polystyrene matrix structure. Suitable examples include Purolite® A140, Purolite® A146, Purolite® A111 and Purolite® A133, also available from Purolite Corporation, Bala Cynwyd, Pa.

In some embodiments, the polystyrene matrix structure is cross-linked with divinylbenzene.

Periodically, it is necessary to regenerate the resin component to remove the ionic contaminants retained on the resin. Such regeneration requires a regenerant solution capable of displacing ionic contaminants from the ionic exchange resin. Methods in the prior art typically require a caustic regenerant solution which is usually made up of sodium hydroxide at a concentration of 4% or 5% (w/v) or even higher. However, Applicants have discovered that significantly lower concentrations of sodium hydroxide of about 1-3% (w/v) are ideal for eluting a significant fraction of ionic contaminants from ion exchange resins, reducing the breakage of resin beads, and reducing fine generation, restoring a high proportion of operating capacity, and allowing for repeated service use of the resin and minimum depreciation in ionic removal performance. Without being bound by any theory of the invention, it is believed that the high efficiency regeneration is achieved by taking advantage of the pH dependent nature of weak base anion exchange resins. At low pH, functional groups of weak base anion exchange resins have a positive charge (e.g., —NH₃⁺) allowing for anion exchange. However, at high pH (i.e., above pH 7) the resin functional groups lose a proton and are converted to the uncharged (e.g., —NH₂) “free-base” form, resulting in complete regeneration.

The regenerant solution may be prepared from diluted solutions of caustic soda. As defined herein, the term “caustic soda” will designate sodium hydroxide (or lye) which is an inorganic compound with the chemical formula NaOH (also written as NaHO). Sodium hydroxide is a white solid and is a highly caustic metallic base alkali salt. It is available in pellets, flakes, granules, and prepared solutions at a number of different concentrations. Sodium hydroxide forms an approximate 50% (by weight) saturated solution with water. Sodium hydroxide is soluble in water, ethanol and methanol. This alkali is deliquescent and readily absorbs moisture and carbon dioxide in air.

As an alternative to a caustic regenerant such as sodium hydroxide, ammonia may be used. Ammonia equilibrates into two forms, NH₄⁺OH⁻ (ionized) and NH₃ (un-ionized). Ammonia will shift to the more favorable un-ionized form to penetrate the hydrophobic shell but shift back to the ionized form when it meets the unregenerated, ionized core. The shell-core effect essentially does not occur and the resin is regenerated homogeneously with minimum stress. However, the use of ammonia as a regenerant in an industrial setting has several disadvantages limiting its use as a suitable regenerant. Ammonia places a high chemical oxygen demand (COD) on waste water treatment plants. In addition, ammonia has several significant health and safety issues, further limiting its use.

In another embodiment, a suitable regenerant for use with the present invention is sodium bicarbonate. Concentrations of sodium bicarbonate solution should ideally be between 3 and 6%.

In some embodiments, the regenerant comprises dilute sodium hydroxide in aqueous solution.

In some embodiments, the regenerant consists essentially of sodium hydroxide in aqueous solution.

As used herein, the term “consists essentially of” (and grammatical variants) means that the regenerant solution comprises no other agents which change the material characteristics of the composition. The term “consists essentially of” does not exclude the presence of other components such as minor impurities, solvents, and the like.

In some embodiments, the regenerant solution comprises up to about 2% sodium hydroxide, or about 2.0, 1.5, 1.0, 0.5, 0.4, 0.3, 0.25, 0.2, 0.125% (w/v) sodium hydroxide.

In some embodiments, the regenerant comprises dilute sodium carbonate in aqueous solution.

In some embodiments, the regenerant consists essentially of sodium carbonate in aqueous solution.

In some embodiments, the regenerant is a dilute solution of sodium carbonate. In some embodiments, the regenerant solution comprises up to about 2% sodium carbonate, or about 2.0, 1.5, 1.0, 0.5, 0.4, 0.3, 0.25, 0.2, 0.125% (w/v) sodium carbonate.

The regeneration step typically reduces the ionic contaminants bound to the resin by at least about 10%, or about 20, 30, 40, 50, 60, 70, 80, 90, 95, or about 99% compared to the amount of ionic contaminants bound to the resin before the regeneration step.

In some embodiments, the regeneration reduces the ionic contaminants bound to the resin by at least 90% or more.

In some embodiments, the regeneration reduces the ionic contaminants bound to the resin by at least 70% or more.

In some embodiments, the regeneration reduces the ionic contaminants bound to the resin by at least 50% or more.

In some embodiments, the regeneration reduces the ionic contaminants bound to the resin by at least 40% or more.

In some embodiments, the regeneration reduces the ionic contaminants bound to the resin by at least 20% or more.

Regeneration may be performed continuously on a portion of the resin removed from the resin bed while ion exchange continues with the remainder of the resin followed by recycling of the regenerated resin. Alternatively, regeneration may be performed during periodic shutdown of the resin bed. In some embodiments, at least one pair of ion exchange columns are loaded with the same volumes of resin with one ion exchange column in service while the other column is off-line and being regenerated with the regenerant solution.

Conventional processing conditions, such as the frequency of regeneration, concentration of the regenerant streams and ratio of regenerant to caustic soda, may vary to a significant extent depending upon the type of feedstock to be processed.

On passage of the feedstock through the resin bed, ionic contaminants are displaced. The resins can either be operated in co-flow mode, with the feedstock and regenerant solution entering and exiting the ion exchange vessel in the same direction, or in counter-flow mode, with feedstock, water and regenerant entering the vessel in opposite directions. Counterflow and co-flow operations will produce similar results and are each suitable for use in the present invention.

In some embodiments, the inventive method reduces the concentration of ionic contaminants in the feedstock by at least 10% or more. In some embodiments, the purification process reduces the concentration of ionic contaminants by at least 15, 20, 25, 30, 50, 75, or 95% or more. In some embodiments, the purification process reduces the concentration of ionic contaminants by at least 90% or more.

In some embodiments, the method reduces the concentration of ionic contaminants in the feedstock by at least 90% or more.

In some embodiments, the method reduces the concentration of ionic contaminants in the feedstock by at least 80% or more.

In some embodiments, the method reduces the concentration of ionic contaminants in the feedstock by at least 70% or more.

In some embodiments, the method reduces the concentration of ionic contaminants in the feedstock by at least 50% or more.

Unless defined otherwise, all technical and scientific terms used herein generally have the same meaning as commonly understood by one of ordinary skill in the art to which this invention belongs. Generally, the nomenclatures used herein are those well-known and commonly employed in the art. The techniques and procedures are generally performed according to conventional methods in the art and various general references. The nomenclature used herein and the procedures in water purification and polymer chemistry described herein are those well-known and commonly employed in the art.

The term “about” or “approximately” means within an acceptable error range for the particular value as determined by one of ordinary skill in the art, which will depend in part on how the value is measured or determined—e.g., the limitations of the measurement system, or the degree of precision required for a particular purpose. For example, “about” can mean within 1 or more than 1 standard deviations, as per the practice in the art. Alternatively, “about” can mean a range of up to 20%, preferably up to 10%, more preferably up to 5%, and more preferably still up to 1% of a given value. Where particular values are described in the application and claims, unless otherwise stated, the term “about” meaning within an acceptable error range for the particular value should be assumed.

As used herein and in the appended claims, the singular forms “a,” “an,” and “the,” include plural referents unless the context clearly indicates otherwise. Thus, for example, reference to “a molecule” includes one or more of such molecules, “a resin” includes one or more of such different resins and reference to “the method” includes reference to equivalent steps and methods known to those of ordinary skill in the art that could be modified or substituted for the methods described herein. As used herein, all concentrations expressed as percentages are measured as weight per volume (w/v), unless otherwise noted.

All U.S. patents and published applications and other publications cited herein are hereby incorporated by reference in their entirety. In the case of conflict or inconsistency, the present disclosure controls.

### EXAMPLES

### Example 1

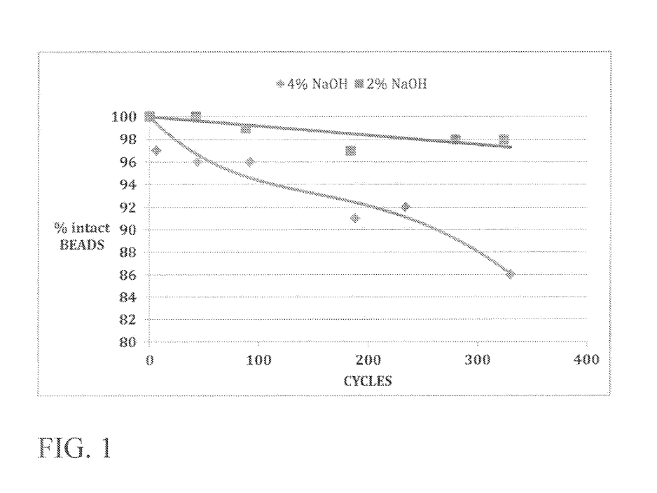

A sample of commercially produced glycolic acid contaminated with approximately 1-2% (w/v) sulfuric acid was subjected to accelerated cycles of a treatment step followed by a regeneration step using a resin bed of an acrylic resin (Purolite A-847). The resin bed was subject to the following treatment: 10 minutes of exposure to the contaminated glycolic acid at a flow rate of 1 bed volume (BV)/hour; 5 minutes of rinse with demineralized water; 10 minutes of exposure to sodium hydroxide regenerant solution at either 2% or 4% (w/v) concentration; followed by a final 5 minute rinse with demineralized water. This cycle was repeated and periodically samples of the resin were taken for analysis of the percentage of intact resin beads remaining. As shown in FIG. 1, at cycle zero, 100% of the beads were intact. For the treatment with the 4% sodium hydroxide solution, bead integrity diminished with the number of cycles of treatment. At 100 cycles approximately 96% of the beads remained intact. At 200 cycles approximately 92% of the beads remained intact; and at 300 cycles 86% of the beads remained intact. In contrast, the same cycling treatment using 2% sodium hydroxide solution resulted in 99% intact beads at 100 cycles; about 98% intact beads after 200 cycles and about 98% intact beads after 300 cycles.

### Example 2

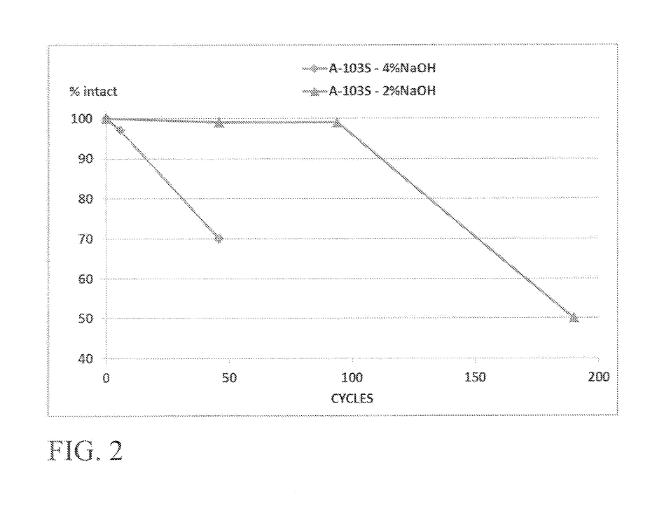

The experiment of Example 1 was repeated, except instead of Purolite A-847 acrylic resin, Purolite A-103S styrenic resin was used. As shown in FIG. 2, at zero cycles 100% of the resin beads were intact. For the beads subjected to a 4% (w/v) sodium hydroxide solution regeneration treatment, after 50 cycles, 70% of the beads remained intact. For the beads subjected to a 2% sodium hydroxide solution regeneration treatment, after 50 cycles almost 100% of the beads remained intact; after 100 cycles almost 100% of the beads remained intact; and after almost 200 cycles about 50% of the beads remained intact.

### Example 3

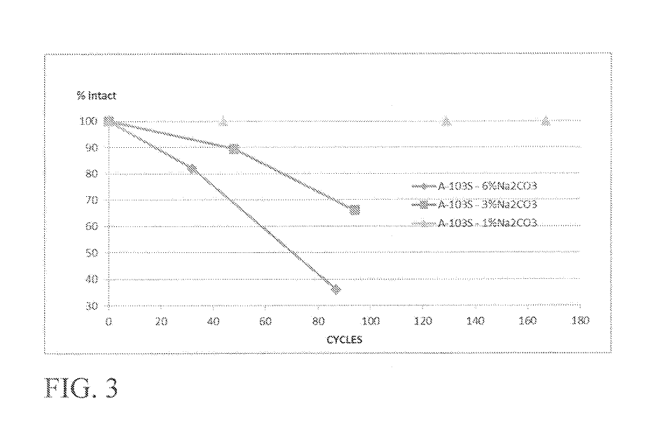

The experiment of Example 2 was repeated, except that instead of a sodium hydroxide solution, sodium carbonate (Na₂CO₃) solution was used as the regenerant. As shown in FIG. 3 at zero cycles 100% of the resin beads were intact. For beads subjected to 6% (w/v) Na₂CO₃ solution regeneration treatment, after about 30 cycles, about 82% of the beads remained intact; after more than 80 cycles, about 35% of the beads remained intact. For the beads subjected to a 3% (w/v) Na₂CO₃ solution regeneration treatment, after about 45 cycles, about 90% of the beads remained intact; after more than 90 cycles, more than 65% of the beads remained intact. For the beads subjected to a 1% (w/v) Na₂CO₃ solution regeneration treatment, after about 40 cycles, about 100% of the beads remained intact; after more than 120 cycles, about 100% of the beads remained intact; and after about 160 cycles about 100% of the beads remained intact.

### Example 4

A synthetic syrup solution was prepared from white table sugar and demineralized water to a concentration of 50-51 Brix (Bx) acidified to 50 meq/l total acidity, using four different acids (15 meq/l HCl; 15 meq/L H₂SO₄; 10 meq/L lactic acid; 10 meq/L acetic acid), and subjected to a 45° C. service run at three bed volumes per hour to a breakthrough of 4.5 and 4.0 pH for three cycles.

Each WBA resin bed was first conditioned as follows:

A column (D×H=35 mm×600 mm) was filled with 200 ml WBA anion resin (in supplied FB form). The column was backwashed for 30 minutes at 50-75% bed expansion with demineralized water. The column was exhausted by passage of 400 ml (2 BV) of 6% HCl at 2 BV/h (6.7 ml/min), followed by a rinse with demineralized water to a conductivity of 100 μS/cm or less (microsiemens/centimeter—a measurement of conductivity indicating purity). The column was then regenerated with 400 ml (2 BV) of 4% (w/v) NaOH at 2 BV/h (6.7 ml/min). A displacement rinse was then carried out with 400 ml (2 BV) of demineralized water at 2 BV/h (6.7 ml/min), followed by a fast rinse at 10 BV/h (33.3 ml/min) to a conductivity end-point of 10 uS/cm.

The regeneration was conducted with 80 g/L (grams of 100% NaOH per liter of resin) NaOH as well as with 64 g/L NaOH dosage, varying the regenerant concentration: 2% vs 3% vs 4% (w/v) NaOH for the same dosage and same flow rate. The influence of regeneration contact time was also studied in case of 2% NaOH and 4% (w/v) NaOH solution.

After each sweetener service run the resin was first sweetened off (by displacing the sweetener from the column using water with 600 ml (3 BV) with demineralized water at a flow rate of 3 BV/h (10 ml/min). Regenerant was then applied by using 400 ml (2 BV) of 4% (w/v) NaOH at 2 BV/h (6.7 ml/min) at 45° C.; or 800 ml (4 BV) of 2% (w/v) NaOH at 2 BV/h (6.7 ml/min) at 45° C. After the regeneration step, the column was subjected to a displacement rinse with 400 ml (2 BV) of demineralized water at 2 BV/h (6.7 ml/min) at 45° C., and a fast rinse at 10 BV/h (33.3.ml/min) to an end point of 10 uS/cm.

For each service run (cycle), effluent samples were collected each 3 BV and close to the end-point, each 1 BV and measured: pH, conductivity, the exact volume; density at 20° C., based on which were calculated: the mass of syrup (kg), brix (kg dry sugar) and productivity (tons dry sugar/m³ of resin). Finally for each resin was reported the number of BVs of treated syrup until the two breakthrough points (pH=4.5 and pH=4.0 respectively) were reached. From this information productivity can be calculated and the WBA resin operating capacity (eq/l) for each cycle and average of the 3 cycles determined. Considering that the influence of regenerant concentration impacts only in cycles 2 and 3, the average productivity and operating capacity for cycles 2 & 3 was also reported, as being more representative.

Finally the operating capacity was reported to the total volume capacity and correlated with the regenerant dosage, concentration, contact time and particle size.

The results using Purolite resin A103Plus are shown below in Table 1A:

TABLE 1A

| | | | Treated BVs | | | |
|---|---|---|---|---|---|---|
| | | | BVs | BVs | BVs | BVs |
| | | | to | to | to | to |
| | | | pH = | pH = | pH = | pH = |
| | | | 4.5 | 4 | 4.5 | 4 |
| Resin | Batch | Regenerant | Average | | Average | |
| Name | No. | 80 g/L; 2BV/h | Cycles 1-3 | | Cycles 2-3 | |
| A 103Plus | 167Q/12/5 | 2% NaOH | 26 | 27.4 | 26.7 | 28.2 |
| A 103Plus | 167Q/12/5 | 3% NaOH | 23.5 | 25.6 | 23.6 | 25.8 |
| A 103Plus | 167Q/12/5 | 4% NaOH | 23.6 | 25.8 | 23.2 | 25.5 |

As can be seen in Table 1A, there was approximately a 10% increase in the number of BVs treated before breakthrough, either at pH 4.5 or 4.0, when using 2% (w/v) NaOH regenerant solution compared to using 3% or 4% (w/v) NaOH regenerant solution, for either the average of cycles 1-3, or the average of cycles 2-3.

Table 1B shows the calculated operating capacity and ratio of operating capacity/total capacity for the experiment conducted with A103Plus resin.

TABLE 1B

| | | | | | Operating capacity/ | |
|---|---|---|---|---|---|---|
| Resin | Batch | Regenerant | Operating capacity, eq/l | | Total capacity | |
| Name | No. | 80 g/L; 2BV/h | Cycles 1-3 | Cycles 2-3 | Cycles 1-3 | Cycles 2-3 |
| A 103Plus | 167Q/12/5 | 2% NaOH | 1.37 | 1.41 | 0.88 | 0.9 |
| A 103Plus | 167Q/12/5 | 3% NaOH | 1.28 | 1.29 | 0.825 | 0.83 |
| A 103Plus | 167Q/12/5 | 4% NaOH | 1.29 | 1.28 | 0.83 | 0.82 |

### Example 5

The Experiment of Example 4 was repeated using Purolite Resin A133S (a WBA resin with a higher ion exchange capacity compared to A103S). The results are shown below in Tables 2A and 2B.

TABLE 2A

| | | | Treated BVs | | | |
|---|---|---|---|---|---|---|
| | | | BVs | BVs | BVs | BVs |
| | | | to | to | to | to |
| | | | pH = | pH = | pH = | pH = |
| | | | 4.5 | 4 | 4.5 | 4 |
| Resin | Batch | Regenerant | Average | | Average | |
| Name | No. | 80 g/L; 2BV/h | Cycles 1-3 | | Cycles 2-3 | |
| A133S | 128T/13/5 | 2% NaOH | 27.6 | 29.3 | 28.9 | 30.7 |
| A133S | 128T/13/5 | 3% NaOH | 25.3 | 27.7 | 25.6 | 28 |
| A133S | 128T/13/5 | 4% NaOH | 25.8 | 26.9 | 25.7 | 27 |

TABLE 2B

| | | | | | Operating capacity/ | |
|---|---|---|---|---|---|---|
| Resin | Batch | Regenerant | Operating capacity, eq/l | | Total capacity | |
| Name | No. | 80 g/L; 2BV/h | Cycles 1-3 | Cycles 2-3 | Cycles 1-3 | Cycles 2-3 |
| A133S | 128T/13/5 | 2% NaOH | 1.47 | 1.54 | 0.86 | 0.9 |
| A133S | 128T/13/5 | 3% NaOH | 1.38 | 1.4 | 0.81 | 0.82 |
| A133S | 128T/13/5 | 4% NaOH | 1.35 | 1.35 | 0.79 | 0.79 |

As can be seen in Table 2A, there was approximately a 10% increase in the number of BV's treated before breakthrough, either at pH 4.5 or 4.0, when using 2% (w/v) NaOH regenerant solution compared to using 3% or 4% (w/v) NaOH regenerant solution, for either the average of cycles 1-3, or the average of cycles 2-3.

### Example 6

The influence of contact time for the same regenerant dosage (80 g/L) was studied for 2% and 4% (w/v) NaOH for WBA resin Purolite A103S Plus (bt. 167Q/12/5), having a TVC of 1.56 eq/L. For the same regenerant dosage (80 g/L) and concentration: 2% (w/v) NaOH was varied the contact time and consequently the flow rate. The throughput of 3 cycles to pH=4.5 and pH=4 was compared for the same resin, A103SPlus. The operating capacity was calculated. The results are shown below in Tables 3A and 3B.

TABLE 3A

| | | | Treated BVs | | | |
|---|---|---|---|---|---|---|
| | | | BVs to | BVs to | BVs to | BVs to |
| Resin | Batch | Regenerant: | pH = 4.5 | pH = 4 | pH = 4.5 | pH = 4 |
| Name | No. | 80 g/L NaOH - 2% sol. | Average Cycles 1-3 | | Average Cycles 2-3 | |
| A 103Plus | 167Q/12/5 | 2 h contact time (2BV/h) | 26.0 | 27.4 | 26.7 | 28.2 |
| A 103Plus | 167Q/12/5 | 1.5 h contact time (3BV/h) | 25.6 | 27.3 | 26.5 | 27.7 |
| A 103Plus | 167Q/12/5 | 1 h contact time (4 BV/h) | 24.0 | 26.4 | 24.2 | 25.95 |

TABLE 3B

| Resin | Batch | Regeneration: | Operating capacity, eq/L | | Op. cap/Total cap. | |
|---|---|---|---|---|---|---|
| Name | No. | 2% NaOH sol. 80 g/L | Cycles 1-3 | Cycles 2-3 | Cycles 1-3 | Cycles 2-3 |
| A 103Plus | 167Q/12/5 | 2 h contact time (2BV/h) | 1.37 | 1.41 | 0.88 | 0.9 |
| A 103Plus | 167Q/12/5 | 1.5 h contact time (3BV/h) | 1.37 | 1.39 | 0.88 | 0.89 |
| A 103Plus | 167Q/12/5 | 1 h contact time (4BV/h) | 1.3 | 1.32 | 0.85 | 0.83 |

As can be seen from the Tables above, using the same concentration of NaOH regenerant (2% w/v), but varying the contact time, as expressed by flow rate in BV/h, increasing the contact time from one hour, to 1.5 hours or 2 hours, increased both the operating capacity of the regenerated resin and the ratio of operating capacity to total capacity of the resin.

### Example 7

Example 6 was repeated using WBA resin Purolite A133S, instead of A103APlus. The results are shown below in Tables 4A and 4B.

TABLE 4A

| | | | Treated BVs | | | |
|---|---|---|---|---|---|---|
| | | | BVs to | BVs to | BVs to | BVs to |
| Resin | Batch | Regenerant: | pH = 4.5 | pH = 4 | pH = 4.5 | pH = 4 |
| Name | No. | 80 g/L NaOH - 2% sol. | Average Cycles 1-3 | | Average Cycles 2-3 | |
| A133S | 128T/13/5 | 2 h contact time (2 BV/h) | 27.6 | 29.3 | 28.9 | 30.7 |
| A133S | 128T/13/5 | 1.5 h contact time (3BV/h) | 26.7 | 29.2 | 26.9 | 28.8 |
| A133S | 128T/13/5 | 1 h contact time (4BV/h) | 25.9 | 27.2 | 26.6 | 28.1 |

TABLE 4B

| Resin | Batch | Regenerant: | Operating capacity, eq/L | | Op. capacity/Total capacity | |
|---|---|---|---|---|---|---|
| Name | No. | 80 g/L NaOH - 2% sol. | Cycles 1-3 | Cycles 2-3 | Cycles 1-3 | Cycles 2-3 |
| A133S | 128T/13/5 | 2 h contact time (2BV/h) | 1.47 | 1.54 | 0.86 | 0.9 |
| A133S | 128T/13/5 | 1.5 h contact time (3BV/h) | 1.46 | 1.44 | 0.86 | 0.85 |
| A133S | 128T/13/5 | 1 h contact time (4BV/h) | 1.36 | 1.4 | 0.8 | 0.83 |

Again, as can be seen from the Tables above, using the same concentration of NaOH regenerant (2% w/v), but varying the contact time, as expressed by flow rate in BV/h, increasing the contact time from one hour, to 1.5 hours or 2 hours, increased both the operating capacity of the regenerated resin and the ratio of operating capacity to total capacity of the resin.

1. A method of regenerating a weak base anion exchange resin, comprising:
providing a weak base anion exchange resin, at least partially bound to acid;
contacting said exchange resin with a regenerant solution, said regenerant solution comprising one or more alkali compounds at a concentration of 3 percent (weight per volume) or less, thereby displacing said acid from said resin; and
rinsing said exchange resin to remove said regenerant solution and said acid from said resin.

2. The method of claim 1, wherein said regenerant solution is contacted with said exchange resin at a flow rate of about 2 bed volumes per hour or less.

3. The method of claim 1, wherein said exchange resin is selected from the group consisting of a gel-type anion exchange resin comprising an acrylic matrix, a macroporous resin with a polystyrene matrix structure, a gel polystyrene matrix structure and a phenol formaldehyde matrix structure.

4. The method of claim 1, wherein said exchange resin is contacted with said regenerant solution until at least 80% of the operating capacity of said resin is restored.

5. The method of claim 1, wherein said alkali compounds are selected from the group consisting of sodium hydroxide, sodium carbonate and mixtures thereof

6. (canceled)

7. The method of claim 1, wherein said alkali compound is sodium hydroxide and said regenerant solution has a concentration of at least 0.25 percent (weight per volume).

8. (canceled)

9. The method of claim 1, wherein said alkali compound is sodium carbonate and said regenerant solution has a concentration of at least 0.25 percent (weight per volume).

10. A method for removing acid from a solution of starch-based sweetener, comprising:
contacting a sweetener solution containing acid with a weak base anion exchange resin to bind said acid to said resin;
removing said exchange resin from contact with said sweetener solution when the capacity of said resin is reduced to a predetermined condition;
contacting said exchange resin having reduced capacity with a regenerant solution, said regenerant solution comprising one or more alkali compounds at a concentration of about 2 percent (weight per volume) or less, thereby displacing said acid from said resin; and
rinsing said exchange resin to remove said regenerant solution and said acid from said resin.

11. The method of claim 10, wherein said regenerant solution is contacted with said exchange resin at a flow rate of about 2 bed volumes per hour or less.

12. The method of claim 10, wherein said exchange resin is selected from the group consisting of a gel-type anion exchange resin comprising an acrylic matrix, a macroporous resin with a polystyrene matrix structure, a gel polystyrene matrix structure and a phenol formaldehyde matrix structure.

13. The method of claim 10, wherein said exchange resin is contacted with said regenerant solution until at least 80% of the operating capacity of said resin is restored.

14. The method of claim 10, wherein said alkali compounds are selected from the group consisting of sodium hydroxide, sodium carbonate and mixtures thereof

15. (canceled)

16. The method of claim 10, wherein said alkali compound is sodium hydroxide and said regenerant solution has a concentration of at least 0.25 percent (weight per volume).

17. The method of claim 10, wherein said alkali compound is sodium carbonate.

18. The method of claim 10, wherein said alkali compound is sodium carbonate and said regenerant solution has a concentration of at least 0.25 percent (weight per volume).

19. The method of claim 10, wherein said acid is present in said starch-based sweetener at a concentration of less than 1 percent (weight per volume).

20. The method of claim 10, wherein said starch-based sweetener is selected from the group consisting of glucose syrup, dextrose, high fructose corn syrup, hydrogenated sweetener, cellulose hydrolyzate and gelatin.

21. The method of claim 10, wherein said acid removed using said resin is selected from the group consisting of sulfuric acid, nitric acid, phosphoric acid, hydrochloric acid and amino acid.

22. A method for removing acid from a solution of organic feedstock, comprising:
contacting an organic feedstock containing acid with a weak base anion exchange resin to bind said acid to said resin;
removing said exchange resin from contact with said organic feedstock when the capacity of said resin is reduced to a predetermined condition;
contacting said exchange resin having reduced capacity with a regenerant solution, said regenerant solution comprising one or more alkali compounds at a concentration of about 2 percent (weight per volume) or less, thereby displacing said acid from said resin; and
rinsing said exchange resin to remove said regenerant solution and said acid from said resin.

23. The method of claim 22, wherein said organic feedstock is selected from the group consisting of acetic acid, citric acid, formic acid, glycolic acid, lactic acid and succinic acid.
